(19) United States
(12) Patent Application Publication
(10) Pub. No.: US 2019/0070315 A1
(43) Pub. Date: Mar. 7, 2019

(54) LOCALIZED IMMUNOSUPPRESSION VIA OPTOGENETICALLY CONTROLLED CELLS

(71) Applicant: UNIVERSITY OF WYOMING, Laramie, WY (US)

(72) Inventors: Jared BUSHMAN, Laramie, WY (US); Mark GOMELSKY, Laramie, WY (US)

(21) Appl. No.: 16/119,934

(22) Filed: Aug. 31, 2018

Related U.S. Application Data
(60) Provisional application No. 62/553,711, filed on Sep. 1, 2017.

(51) Int. Cl. A61K 48/00; C07K 16/44; A61K 41/00; C12N 5/0783; C12N 5/0775; C12N 9/02; C12N 9/16; C07K 14/195; C12N 9/12; A61N 5/06

(52) U.S. Cl. CPC A61K 48/0083; A61N 2005/0659; A61K 41/0057; C12N 5/0637; C12N 5/0667; C12N 9/0071; C12Y 114/14; C12Y 301/04; C12N 9/16; C07K 14/195; C12N 9/1247; C12Y 207/07006; A61N 5/062; C07K 2319/00; C12N 2510/00; C07K 2319/70; C07K 2319/80; A61N 2005/0663; C07K 16/44

(57) ABSTRACT

Embodiments described herein relate to suppressing the immune response locally within tissue transplants and certain conditions improperly affecting the immune system using optogenetically controlled cells. More specifically, embodiments described herein provide for localized immunosuppression surrounding tissue transplants and illness locations as an alternative to systemically suppressing a patient's entire immune system. Methods include implantation of optogenetically modified immunosuppressive cells that are configured to alter their biological activity to enhance their immunosuppressive activity in response to exposure of wavelengths of light in the red and near-infrared window spectral region (620-900 nm).

### CROSS-REFERENCE TO RELATED APPLICATIONS

This application claims benefit of U.S. Provisional Patent Application No. 62/553,711, filed Sep. 1, 2017, the entirety of which is herein incorporated by reference.

### GOVERNMENT RIGHTS

This invention was made with government support under W81XWH-17-1-0402 awarded by the Department of Defense. The government has certain rights in the invention.

### BACKGROUND

### Field

Embodiments of the present disclosure generally relate to methods and materials for suppressing the immune system in a localized manner.

### Description of the Related Art

Tens of millions of individuals in the United States suffer from conditions resulting from the improper and/or undesirable activity of the immune system, such as psoriasis, rheumatoid arthritis, multiple sclerosis, graft versus host disease, and rejection of tissue transplants. In the vast majority of these conditions, the immune system is not targeting all of the body's tissues but is rather improperly or undesirably active at specific locations on the patient's body. One approach currently used to treat such patients includes immunosuppressive therapy that is administered and acts on a systemic, whole body level. However, immunosuppressive drugs and interventions have dangerous side effects (e.g. nephrotoxicity, infectious diseases, cancer) and can put the patients at increased risk of contracting infectious diseases.

Cell therapy, which is the implantation of cells for treatment of health conditions, is another promising and growing approach being utilized to help treat such conditions of improper and/or undesirable activity of the immune system. Many clinical trials have been conducted with cell therapy, and several cell therapy methods have been approved by regulatory agencies for use while many more are in the clinical and pre-clinical stages of investigation. One of the shortcomings of cell therapy is that once the cells are implanted in the patient, the clinicians and patient have no control over those cells. This is problematic, as the cells may prematurely die, be eliminated by the immune system, migrate away from the intended site of activation, differentiate into a non-beneficial cell type, and/or alter their function in a way that is either no longer beneficial to the patient and may be deleterious. Loss or alteration of the cells at the intended site of activity impairs the therapeutic effect of the cell therapy. In the case of immunosuppressive cells, such cells may create immunosuppressive conditions at locations other than the desired sites of activity.

Thus, what is needed in the art are improved methods and materials capable of suppressing the immune system in a controlled and localized manner.

### SUMMARY

In one embodiment, a method of generating optogenetically controlled cells includes modifying immunosuppressive cells or precursors of immunosuppressive cells to comprise an optogenetic system, the optogenetic system being configured to express one or more genetic elements in the cell, implanting the modified cells into a patient, and applying light of a certain wavelength to a localized area of the patient to control the biological behavior of the cells.

In another embodiment, a method of optogenetically modifying cells includes binding a chromophore to a protein in immunosuppressive cells, the protein comprising a photosensory domain and an enzymatic domain, applying light of a certain wavelength to the cells to increase enzyme activity in the cells, the increased enzyme activity producing cyclic adenosine monophosphate in the cells, placing cyclic adenosine monophosphate responsive elements in a promoter sequence of the cells, and binding cyclic adenosine monophosphate sensitive transcription factors to the cyclic adenosine monophosphate responsive elements to alter expression of one or more genetic elements.

In yet another embodiment, a method of optogenetically modifying cells includes extracting immunosuppressive cells or stem-like precursors of immunosuppressive cells from a patient, binding a chromophore to a protein in the cells, the protein comprising a photosensory domain and an enzymatic domain, applying light of a certain wavelength to the cells to increase enzyme activity in the, the increased enzyme activity producing cyclic-di-guanosine monophosphate in the cells, placing cyclic-di-guanosine monophosphate responsive elements in a promoter sequence of the cell, and binding cyclic-di-guanosine monophosphate sensitive transcription factors to the cyclic-di-guanosine monophosphate responsive elements to alter expression of one or more genetic elements.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates operations of a method of using optogenetically modified cells as immunosuppressants, according to one embodiment.

FIGS. 2A-2C illustrate a system utilizing cyclic adenosine monophosphate (cAMP) for modifying cells to be optogenetic at various stages, according to one embodiment.

FIGS. 3A-3D illustrate a system utilizing cyclic-di-guanosine monophosphate (c-di-GMP) for modifying cells to be optogenetic at various stages, according to one embodiment.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments described herein relate to suppressing the immune response locally within tissue allografts such as vascularized composite allografts (VCAs), and certain illnesses improperly affecting the immune system using optogenetically controlled immunosuppressive cells. More specifically, embodiments described herein provide for localized immunosuppression surrounding tissue allografts, such as VCAs, and illness locations as an alternative to systemically suppressing a patient's entire immune system. Methods include localized implantation of optogenetically modified immunosuppressive cells that are configured to express one or more genetic elements in response to exposure of certain wavelengths of light.

Localized immunosuppression is defined herein as non-systemic immunosuppression where the entirety or majority of the immune suppression is taking place at a specific body location or locations rather than being distributed throughout the entire body. More specifically, localized immunosuppression is defined herein as suppressing the immune system response in the local environment of an allograft, such as VCA or illness sites, thereby eliminating or minimizing the utilization of systemically delivered immunosuppressive agents. Optogenetically controlled immunosuppressive cells (OCISCs) are defined herein as cells comprising optogenetic cassettes or systems with biological activity to suppress an immune response. The one or more biological activities are enhanced upon irradiation with specific wavelengths of light. For example, OCISCs include regulatory T cells, mesenchymal stromal cells, antigen presenting cells, macrophages, Schwann cells, and stem-like precursors of immunosuppressive cell types, among others.

Methods for delivery of said OCISCs, encompassing all methods for systemic and localized delivery of said OCISCs to the allograft or illness site, include, but are not limited to, vascular infusion, microfluidics, catheterization, and/or injection to deliver agents from internal or external devices to the tissue graft or illness site, implantation of biocompatible biomaterial carriers in local proximity to the allograft or illness, such as, biomaterials including poly(ethylene glycol), poly(lactic acid), poly(lactic-co-glycolic) acid, collagen, and fibrin, among others, that maintain viability of the agents in a desired location and enable release of the agents if desired.

It is contemplated that by targeting the immune response to the graft or site of improper immune system activity, a sufficient confined suppression in the immune response to the allograft or illness site can be achieved. In one embodiment, the systemic application of OCISCs can be utilized to target the immune response to the allograft or illness site. Alternatively, targeting of the immune response is enabled by local application of immunosuppressants instead of systemic application of immunosuppressants. Localized immunosuppression minimizes the risks associated with conventional cell therapy and systemic application of immunosuppressants. For example, local administration of OCISCs leaves the immune response in the rest of the body largely intact. In addition, lower doses of OCISCs administered locally are much less likely to cause kidney damage as comparatively negligible amounts of OCISCs enter the circulatory system. Still further, localized and temporary immunosuppression is much less likely to contribute to causing lymphoproliferative disorders.

It is also contemplated that localized immunosuppression through localized activation of OCISCs will remove or minimize utilization of systemic immunosuppression. For example, instead of daily systemic immunosuppression for VCAs during the initial regeneration period, systemic immunosuppression may be delivered on a less frequent basis, thus, improving the quality of care for the patient and improving prospects for patient compliance.

It is also contemplated that the OCISCs responsible for the localized immunosuppression can be controlled. OCISCs are controlled through optogenetics to activate one or more immunosuppressive mechanisms. The OCISCs may then be implanted at the VCA or illness location and controlled using certain wavelengths of light. For example, the cells may be controlled by exposure to certain wavelengths of light to proliferate, replicate, secrete certain cytokines, myelinate neurons, etc. Broadly, the optogenetically modified cells work by transducing the signal provided by a specific light wavelength into an output than affects cell behavior. It is contemplated that controlling the OCISCs through optogenetics can be accomplished by applying an external light source capable of penetrating through skin, bone, and flesh. Therefore, external light sources may supply light that penetrates through skin, bone, and flesh. Wavelengths of light used may include, but are not limited to, near-infrared optical window (NIRW) light, defined as having wavelengths in the range of 670-900 nm, and red light defined here as having wavelengths of 620-670 nm. Such light is known to be safe for repeated and prolonged administration in humans, and is optimal for penetrating through human tissues. Light sources may emit light in narrow or broad range of wavelengths, including white light that contains wavelengths in the 620-900 nm range.

### Optogenetically Controlled OCISCs

Regulatory T-cells (Tregs) are a sub-population of CD4+ cells and suppress activated effector T-cells through a variety of mechanisms linked to Treg FoxP3 expression. Generally, Tregs function of APCs and effector T-cell populations and proliferate in response to IL-2 and down-regulate the adaptive immune response of several immune cell types. Tregs also attenuate graft versus host disease and a number of other autoimmune disorders. Accordingly, embodiments described herein contemplate local activation of Tregs for suppression of an acquired immune response to tissue transplants and improper immune system activity.

In addition, mesenchymal stromal cells (MSCs) are also an immunosuppressive cell type contemplated for localized activation to suppress the immune response to VCAs and illness sites as MSCs attenuate many autoimmune diseases and suppress the activity of many immune cell types. Schwann cells are further contemplated for localized delivery to suppress the immune response to tissue transplants, injury sites and illness sites, as Schwann cells are believed to stimulate regeneration. Antigen presenting cells (APCs) are further contemplated for localized delivery to suppress the immune response to VCAs and illness sites. Macrophages of M2-type possess specific immunosuppressive properties and may also be used for localized immunosuppression.

### Utilizing Optogenetically Controlled OCISCs

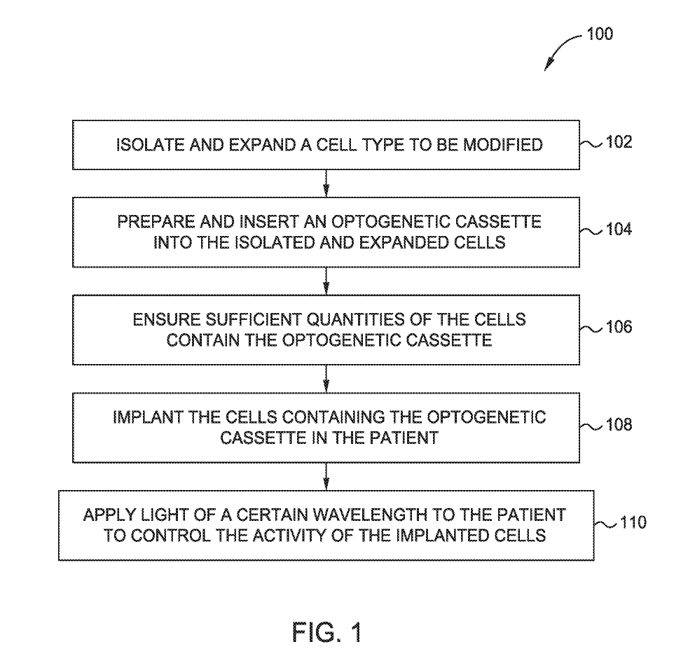

FIG. 1 illustrates a method 100 of using optogenetically modified cells as immunosuppressants, according to one embodiment. Method 100 may be used as part of a treatment for tissue transplant or illnesses that negatively affect the immune system, such as psoriasis, rheumatoid arthritis, multiple sclerosis, graft versus host disease, and rejection of organ transplant, among others. Any of the above described OCISCs may be implemented with method 100.

In operation 102 of method 100, cells of a specific type to be used for immunosuppression are isolated and expanded ex vivo. Any cell type for any purpose may be used, so long as the cells can be irradiated with sufficient light. Cells may be allogeneic, autologous or xenogeneic with respect to the patient. As discussed above, the cell type may include Tregs, MSCs, Schwann cells, APCs, macrophages, and stem-like precursors of these cells types, among others. For Tregs, operation 102 may include taking blood from the patient, isolating the Tregs based on immunophenotype (CD4+, CD25+), and expanding the cell population in vitro. For MSCs, operation 102 may include taking MSCs from a patient's tissue. MSCs can be isolated from many different tissue types, such as bone marrow, adipose tissue, and skeletal tissue. The MSCs may then be expanded in vitro. For Schwann cells, operation 102 may include taking a biopsy of a patient's peripheral nerve and then expanding the cells in vitro. Alternatively, Schwann cells may also be generated from a patient's stem cells.

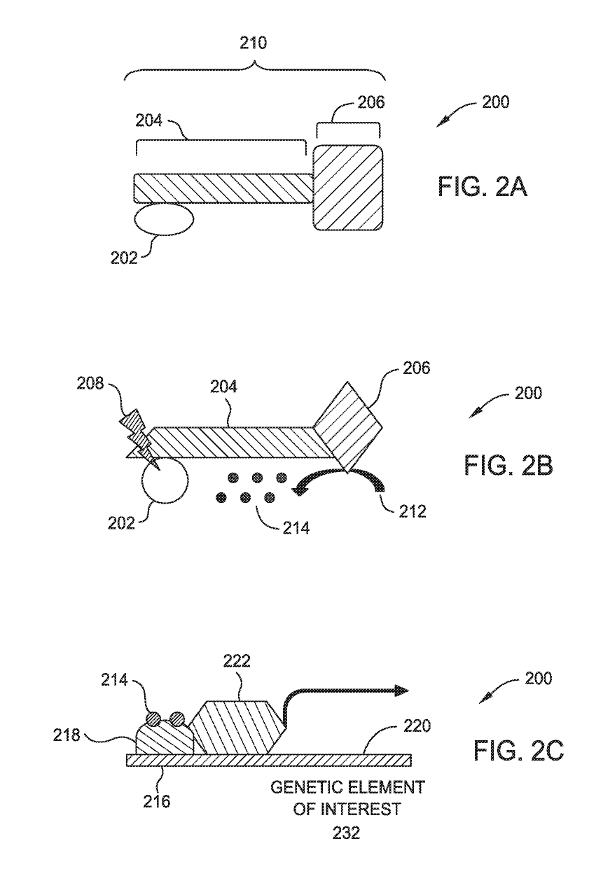

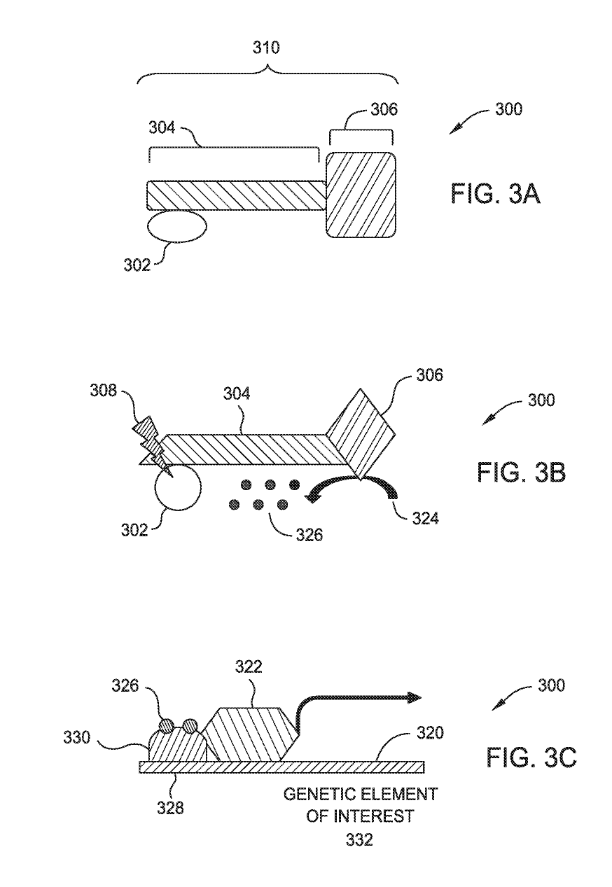

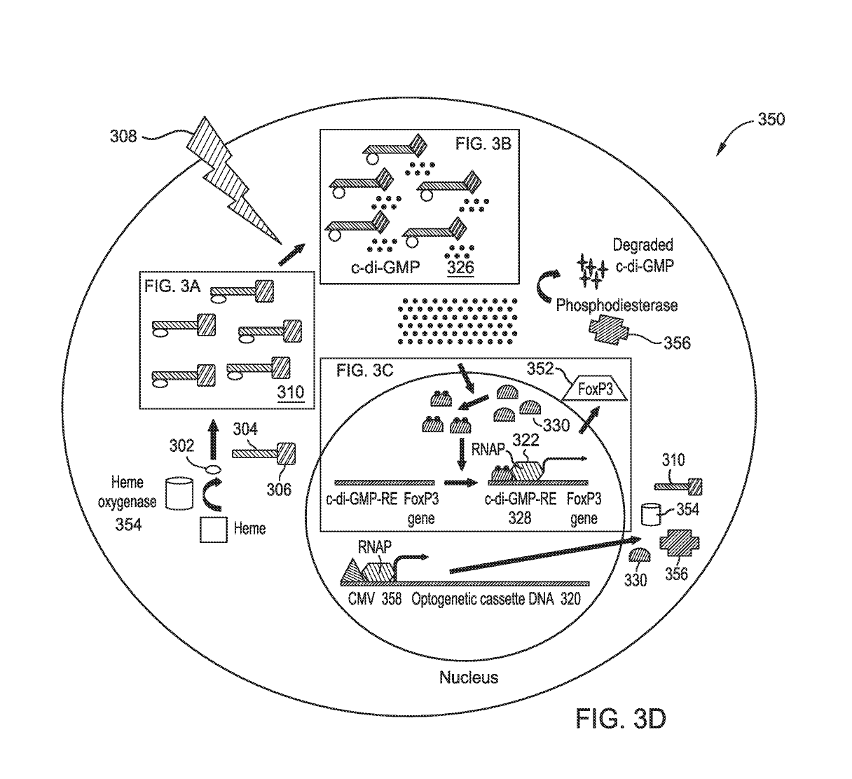

In operation 104, an optogenetic cassette or system is genetically engineered and inserted into the cells. The optogenetic cassette modifies the cells to optogenetically controllable and express desired cellular behaviors. Preparation of the optogenetic cassette is described in further detail below in FIGS. 2A-2C and FIGS. 3A-3D. In one embodiment, the optogenetic cassette may be prepared using the cyclic adenosine monophosphate (cAMP)-based system 200 of FIGS. 2A-2C and/or the system cyclic-di-guanosine monophosphate (c-di-GMP)-based system 300 of FIGS. 3A-3D. Once the optogenetic cassette is genetically engineered, the optogenetic cassette is then inserted into the cells by transfection, viral infection, CRISPR/Cas9, or any other suitable method of DNA delivery, resulting in modified cells containing the optogenetic cassette.

Examples of genetic elements to be optogenetically controlled or expressed through the optogenetic cassette may include, but are not limited to, activation of genetic elements controlling cell proliferation or activation of desirable cellular activities. By controlling the proliferation of the modified cells, a higher density of OCISCs is generated at the intended location without the need for systemic immunosuppression. As such, sufficient quantities of the modified cells which include the cassettes can be maintained at the desired site. Controlling the activation of the modified cells which include the optogenetic cassette permits the cells to be activated and de-activated as needed. For example, some illnesses such as arthritis may not require the modified cells to be constantly active or to actively suppress the immune system at all times.

It is contemplated that any of the genes found in mammals may be selected for the optogenetic cassette to control various cellular functions; however genes may be specific to the type of cell used. In one example, cell death is a cellular function which can be controlled optogenetically. In this example, the cells may be genetically engineered such that optogenetic control of the cells causes cell death. It is believed this programmable cell death may be advantageous should the therapy exhibit maleficence so that the cells can be eliminated.

In operation 106, the modified OCISCs comprising the optogenetic cassette are selected to ensure sufficient quantities of the cells contain the optogenetic cassette. A variety of methods may be implemented to ensure that a sufficient quantity of the cells contain the optogenetic cassette. For example, if a fluorescent protein, such as green fluorescent protein (GFP), is selected as the genetic element to be optogenetically expressed and placed in the cassette, the modified cells may be sorted to identify the GFP positive cells. Additional methods for ensuring that a sufficient quantity of cells contains the optogenetic cassette include drug selection, such as resistance to antibiotics neomycin or puromycin, and surface markers, among others. In one embodiment, operation 106 is optional. If it is determined that there is an insufficient quantity of the cells containing the optogenetic cassette in operation 106, operations 102, 104, and/or 106 may be repeated one or more times.

In operation 108, the modified cells comprising the optogenetic cassette are implanted into the patient. The patient may be human or animal. The cells may be implanted through systemic injecting into the bloodstream (vascular infusion), or an injection into the target tissue with or without a pharmaceutically acceptable carrier. The cells may be implanted into a localized area of the patient, such as at the tissue transplant or illness site. For example, if a patient has had a limb transplant, the cells would be implanted into the transplanted limb and surrounding area.

In operation 110, light of a certain wavelength is applied to control or express the genetic elements of the optogenetic cassette of the implanted cells in vivo. Applying the light of a certain wavelength enables regulation of the desired cell processes in the implanted OCISCs in vivo. Such processes may include gene transcription or the increasing of cells signaling molecules, such as cyclic adenosine monophosphate or cyclic-di-guanosine monophosphate, with or without transcriptional control. The wavelength of light applied is a predetermined wavelength engineered to optogenetically control or express the selected genetic elements of the cell. The optogenetic cassette is prepared in operation 104 to be responsive to predetermined wavelengths of light.

Light may be applied using any suitable device, and may be applied to the implanted cells either externally or internally, i.e. implanted in the body. In at least one implementation, the applied light is red light (defined here as 620-670 nm) or NIRW light (defined here as 670-900 nm). Red and NIRW light penetrates skin, bone, and flesh, and is safe for repeated and prolonged administration. A light emitting device, such as a red light or NIRW-emitting device, may be placed close to the skin of the cell implantation area, or area where the OCISCs to be controlled are located, repeatedly over a period of time. For example, a patient may apply red or NIRW light to the implanted cells one or more times a day for any length of time. In one embodiment, the light emitting device, may be implanted in the patient. In such an embodiment, the implanted devices may include a timer for determining when and for how long the light will be applied, or the implanted devices may be controlled via another device, such as a computer.

Since the genetic elements of the cells are controlled using light, method 100 may be considered a virtually pain free, non-invasive method, and patients may self-treat at home or in a clinical setting with ease once the cells are implanted. Additionally, depending on the type of illness or tissue transplant being treated and the cellular process being expressed or controlled, a patient may have the optogenetically controlled cells implanted infrequently. For example, if the expressed genetic element proliferates the modified cells at the implantation site in vivo, sufficient quantities of the OCISCs may remain active at the site. Since the patient may have the ability to proliferate the modified cells at home using light, the patient may not require more frequent cell implantations, regardless of whether some of the modified cells migrate away from the site, become permanently inactive, or die. Furthermore, localized immunosuppression can be achieved, as the modified cells are implanted at sites exhibiting undesirable immune system activity or sites which would benefit from localized immunosuppression.

### Cellular Optogenetic Modification

Embodiments described herein provide for methods of modifying cells to include optogenetic systems or cassettes. The modified cells may be any of the immunosuppressive cells discussed above, such as Tregs, MSCs, APCs, Schwann cells, M2 macrophages, or stem-like precursors of immunosuppressive cells.

To control the cells responsible for the localized immunosuppression, the cells are engineered or modified to include an optogenetic system prior to implantation of the cells into the patient. Generally, the optogenetic system relies on three main components: first, a protein that is responsive to certain wavelengths of light being produced by the modified cells, where applying light induces a change in the conformation and activity of the protein; second, the light-induced change in conformation of this protein leads to a biologically-interpretable stimulus in the cell, thus translating light into a biological output signal; and third, the optogenetically responsive cells include mechanisms to translate the biological signal into an alteration in the expression or activity of desired genetic elements. In some cells the production of the biologically-interpretable stimulus may alone be sufficient to control the cell behavior as desired while other circumstances it may be optimal for the system to include the genetic elements under transcriptional control of the biologically-interpretable stimulus.

FIGS. 2A-2C illustrate a system 200 utilizing cyclic adenosine monophosphate (cAMP) as the biologically-interpretable stimulus for modifying cells to be optogenetically responsive at various stages, according to one embodiment. The system 200 may be used in combination with method 100, such as to prepare the optogenetic cassette in operation 104. Furthermore, while FIGS. 2A-2C illustrate only one cell, the system 200 may be used to modify a plurality of cells.

In FIG. 2A, a protein 210 is produced or inserted into a cell in vitro. The protein 210 comprises a photosensory domain 204 and an enzymatic domain 206. A chromophore 202, which is involved in light sensing, is produced by the cell and bound to the photosensory domain 204 of the protein 210. The chromophore 202 is sensitive to certain wavelengths of light, such as red (620-670 nm) and NIRW light (670-900 nm). The chromophore 202 may be biliverdin IXa, which is a product of heme turnover. Animal and human cells produce biliverdin IXa, and biliverdin IXa absorbs light in wavelengths of red and NIRW spectrum. The protein 210 has high affinity for biliverdin IXa and may bind biliverdin IXa covalently or noncovalently.

FIG. 2B illustrates the conformational changes of the protein 210 due to the application of certain wavelengths of light 208. When certain wavelengths of light 208 reach the chromophore 202, the chromophore 202 absorbs light, which results in a conformational change in the chromophore 202. The protein components within the photosensory domain 204 are sensitive to conformational changes in the chromophore 202, so that when the chromophore 202 changes conformation in response to certain wavelengths of light 208, the photosensory domain 204 also changes conformation.

The conformational change in the photosensory domain 204 leads to an increase in the activity of the enzymatic domain 206. For the system 200, the enzymatic domain 206 on the protein 210 has low activity in the absence of light 208. However, light induced conformational changes in the photosensory protein domain 204 result in a conformational change in the enzymatic domain 206. The conformational change induced in the enzymatic domain 206 increases the activity of an enzyme, such as adenylate cyclase, in the enzymatic domain 206. For the system 200, the adenylate cyclase enzyme then catalyzes the production of cAMP 214 from intracellular adenosine triphosphate (ATP) 212.

The cAMP 214 in the system 200 is a biologically interpretable signal produced in response to light. cAMP 214 is a second messenger that functions in many cell types, where the upregulation and downregulation of cAMP 214 levels alters cellular behaviors. For some OCISC types, just the upregulation of cAMP 214 may be sufficient to induce the desirable change in cellular behavior. For other cell types, a method must be utilized to translate the change in cAMP 214 levels into a change in biological activity such as changes in gene expression.

FIG. 2C illustrates translating the change in cAMP 214 levels into a change in gene expression. Many cell types express transcription factors 218 that cAMP 214 affects the activity of, leading to an increase or decrease in gene expression dependent on the level of cAMP 214. Thus, in the cAMP system 200, transcription factors 218 need not be added as an additional component, as the transcription factors 218 are already present in the cells. However, it is contemplated that genetically engineered, synthetic cAMP-dependent transcription factors may be added.

To control expression of genetic elements 232, cAMP-responsive elements (CRE) 216 are placed in the promoter sequence. The cAMP-sensitive transcription factors 218 bind to specific genetic sequences of DNA 220 in the CREs 216 (i.e., the promoters of genes) and differentially recruit RNA polymerase (RNAP) 222, leading to changes in the production of genes or other genetic elements 232. Such changes can then alter cellular behavior. Any gene or genetic element 232 capable of controlling or expressing cellular functions may be selected, such as the genetic elements responsible for activation of cellular function and/or cell proliferation. Alterations in the expression of genetic elements 232 occur as the cAMP sensitive transcription factors 218 bind to the CRE 216. The sequences for genetic elements 232 to be differentially regulated by the system 200 are constructed and synthetically placed in such a way that when the cAMP sensitive transcription factors 218 bind to the CRE 216, transcription of the genetic elements 232 is increased.

Genetically engineered, synthetic cAMP-dependent transcription factors may be designed to bind the CRE 216 element or another DNA sequence. Sequence specificity may be encoded in the known DNA-binding factors which may include, but are not limited to, zinc finger proteins, transcription activator and nuclease (TALEN) proteins, CRISPR proteins, or bacterial cAMP-dependent transcription factors such as CRP from E. coli.

FIGS. 3A-3D illustrate a system 300 utilizing cyclic-di-guanosine monophosphate (c-di-GMP) for modifying cells to be responsive to light, according to one embodiment. The system 300 may be used in combination with method 100, such as to prepare the optogenetic cassette in operation 104. Furthermore, while FIGS. 3A-3C illustrate only one cell, the system 200 may be used to modify a plurality of cells, as shown in FIG. 3D.

In FIG. 3A, a protein 310 is produced or inserted into a cell in vitro. The protein 310 comprises a photosensory domain 304 and an enzymatic domain 306. A chromophore 302 produced by the cell is bound to the photosensory domain 304 of the protein 310. The chromophore 302 is sensitive to certain wavelengths of light, such as red (620-670 nm) and NIRW light (670-900 nm). The chromophore 302 may be biliverdin IXa, which is a product of heme turnover. Animal and human cells produce biliverdin IXa, and biliverdin IXa absorbs light in wavelengths of red and NIRW spectrum. The protein 310 has a high affinity for biliverdin IXa and may bind biliverdin IXa covalently or noncovalently.

FIG. 3B illustrates the conformational changes of the protein 310 due to the application of certain wavelengths of light 308. When certain wavelengths of light 308 reach the chromophore 302, the chromophore 302 absorbs light, which results in a conformational change in the chromophore 302. The protein components within the photosensory domain 304 are sensitive to conformational changes in the chromophore 302, so that when the chromophore 302 changes conformation in response to certain wavelengths of light 308, the photosensory domain 304 also changes conformation.

The conformational change in the photosensory domain 304 leads to an increase in the activity of the enzymatic domain 306. For the system 300, the enzymatic domain 306 on the protein 310 has low activity in the absence of light 308. However, light induced conformational changes in the photosensory protein domain 304 result in a conformational change in the enzymatic domain 306. The conformational change induced in the enzymatic domain 306 increases the activity of an enzyme in the enzymatic domain 306. For the system 300, the enzyme in the enzymatic domain 306 produces c-di-GMP 326 from intracellular guanosine triphosphate (GTP) 324. Because c-di-GMP 326 is not a molecular entity found in mammalian cells (in contrast to cAMP 214 of the system 200 of FIGS. 2A-2C), additional components are included in the system 300 to translate the production of c-di-GMP 326 into changes in the production of genetic elements and to degrade c-di-GMP for the purpose of terminating signal transduction.

FIG. 3C illustrates translating the production of c-di-GMP 326 into changes in the production of genetic elements. An additional component utilized in the system 300 is the production of a transcription factor 330 whose DNA binding depends on the presence of c-di-GMP 326. Since c-di-GMP 326 is not present in mammalian cells, c-di-GMP sensitive transcription factors 330 are inserted into and produced in the cells used with the system 300. Numerous c-di-GMP sensitive transcription factors 330 are able to translate the production of c-di-GMP 326 into changes in gene expression, examples of which include, but are not limited to BldD from Streptomyces species, Clp from Xanthomonas, and MrkH from Klebsiella, among others.

The transcription factor 330 differentially binds to a sequence of DNA 320, termed a c-di-GMP response element (c-di-GMP-RE) 328. To control expression of genetic elements, c-di-GMP-REs 328 are placed in the promoter sequence. Any gene or genetic element capable of controlling or expressing cellular functions may be selected, such as the genetic elements responsible for activation of cellular function and/or for cell proliferation. Thus, when certain wavelengths of light are applied, the production of c-di-GMP 326 is increased, c-di-GMP 326 binds to the transcription factor 330, which then binds to c-di-GMP-REs 328 in DNA 320 and recruits RNAP 318 to induce changes in gene expression. Such changes can then alter cellular behavior. Alterations in the expression of genetic elements occur as the c-di-GMP sensitive transcription factors 330 bind to the c-di-GMP-RE 328. The sequences for genetic elements 332 to be differentially regulated by the system 300 are constructed and synthetically placed in such a way that when the c-di-GMP sensitive transcription factors 330 bind to the c-di-GMP-RE 328, transcription of the genetic elements 332 is increased. It is also contemplated that the production of c-di-GMP is able to modify cellular behavior independently of controlling transcription.

Both systems 200, 300 may utilize additional components to tune the optogenetic system 200, 300 for maximal activation. For example, the systems 200, 300 may include a heme oxygenase protein (several varieties exist) to increase production of chromophores, such as biliverdin IXa, if levels in the cells are too low. The systems 200, 300 may further include phosphodiesterases (several varieties exist) that degrade cAMP 214 or c-di-GMP 326 so as to maintain low basal levels in the absence of light.

FIG. 3D illustrates a cell 350 modified using the c-di-GMP optogenetic system 300. In FIG. 3D, biliverdin IXα is used as the chromophore 302, although other chromophores may be implemented. Furthermore, NIRW light is used as the wavelength of light applied in FIG. 3D, but other light wavelengths may be used as well. Additionally, FoxP3, which helps to regulate the immune system, is used as the genetic element to be controlled or expressed in FIG. 3D, however other genetic elements may be selected as well. It is contemplated that any of the genetic elements found in mammalian cells may be used controlled in the system 300 to control cellular functions; however genes may be specific to the type of cell used.

In FIG. 3D, DNA 320 containing an optogenetic cassette and genetic elements, such as FoxP3 252, to be controlled via certain wavelengths of light 308 are integrated into the cell 350. Components such as the protein 310 comprising the photosensor 304 and the enzyme 306, the transcription factor 330, and optional components heme oxygenase 354 and phosphodiesterase 356, are produced under a constitutively active promoter 358, such as cytomegalovirus (CMV). While CMV is shown in FIG. 3D, a different promoter can be used to ensure the correct quantity of the optogenetic protein, such as protein 310, or other proteins are expressed.

In the cytoplasm of the cell 350, the protein 310 comprising the photosensor 304 and the enzyme 306 binds to biliverdin IXα 302, as described in FIG. 3A above (labeled 3A in FIG. 3D). NIRW light 308 is applied, inducing a conformational change in biliverdin IXα 302, the photosensor 304, and the enzyme 306, and leading to the production of c-di-GMP 326, as described in FIG. 3B above (labeled 3B in FIG. 3D). The c-di-GMP 326 binds with the transcription factor 330 enabling the c-di-GMP 326 to then bind to the c-di-GMP-RE 328 of DNA 320. The c-di-GMP-bound transcription factor 330 recruits RNAP 318 and induces transcription of the target genetic element, resulting in the generation of the FoxP3 protein 352. In this manner, FoxP3 352 production is controlled by NIRW light 308.

Once the systems 200 and 300 have been utilized to modify cells to comprise optogenetically controlled genetic elements, the OCISCs are implanted into the patient, and light is applied to control the cells, such as in operation 108 and 110 of method 100 above. As such, certain wavelengths of light enable transcriptional regulation of desired genetic elements in the implanted cells.

Moreover, method 100 utilized with system 200 and/or system 300 for optogenetic control of cells may also be useful for manipulation of cells outside of animals or humans during in vitro conditions. For example, the optogenetic systems 200, 300 may be used to study the biological mechanisms occurring in cells. In such a case, the optogenetic system 200, 300 inserted in the cells is modified in a way that alters cellular behavior of genetic elements in response to certain wavelengths of light, such as red or NIRW light, administered to the cells in vitro. Another use of method 100 utilized with system 200 and/or system 300 is the use of optogenetic regulation of genetic elements to circumvent cell supplementation with expensive growth factors. In such a case, the optogenetic system 200, 300 regulates genetic elements in cells so that exposure to certain wavelengths of light, such as NIRW light, causes cell proliferation. This could obviate or significantly reduce the costs to grow cells. Another implementation utilizes certain wavelengths of light, such as red or NIRW light, and the optogenetic systems 200, 300 to increase production of biological compounds generated by cells, such as proteins, lipids, glycans, metabolites and/or genetic material. Where a cell is used to produce a protein (such as a growth factor, cytokine, antibody etc.), the expression of the protein (or proteins responsible for producing the biological compound) is placed under optogenetic control, and thus, production of the protein or biological compound is enhanced by exposure to certain wavelengths of light.

The optogenetic systems involving cAMP 214 or c-di-GMP 326 are presented as examples of red and NIRW light-dependent optogenetic systems. Additional red and NIRW light-dependent optogenetic systems, such as a system for NIRW light-dependent protein-protein interactions involving PpsR2 and BphP1 proteins from Rhodopseudomonas palustris, where one of the components is linked to a DNA-binding protein domain and another is linked to a transactivator domain involved in RNA polymerase recruitment, may be advantageously implemented in accordance with the embodiments described herein. Red and NIRW light-dependent optogenetic systems involving second messengers other than cAMP or c-di-GMP, such as cGMP, c-di-AMP, c-di-AGMP (cGAMP), may be engineered and applied in place of the systems illustrated in FIGS. 2 and 3. Other red and NIRW light-dependent optogenetic systems involving light-dependent protein-protein interactions may be engineered and applied in place of the PpsR2-BphP1 system.

By optogenetically controlling or expressing the desired genetic elements, localized immunosuppression may be achieved. Furthermore, using certain wavelengths of light, such as red or NIRW light, for transcriptional regulation of desired genetic elements in cells containing the optogenetic systems or cassettes enables localized immunosuppression in an easy and pain free manner without the risks commonly associated with systemic immunosuppression. As such, improper or undesirable immune system activity can be treated or regulated in a confined manner without prohibiting or limiting the immune system in the rest of the body. Additionally, patients may easily treat improper immune system activity at home, which may reduce costs associated with systemic immunosuppression, such as reducing medical and pharmaceutical bills.

While the foregoing is directed to embodiments of the present disclosure, other and further embodiments of the disclosure may be devised without departing from the basic scope thereof, and the scope thereof is determined by the claims that follow.

What is claimed is:

1. A method of generating optogenetically responsive cells, comprising:
modifying immunosuppressive cells or precursors of immunosuppressive cells to comprise an optogenetic system, the optogenetic system being configured to express one or more genetic elements in the cell;
implanting the modified cells into a patient; and
applying light of a certain wavelength to a localized area of the patient to control biological behavior of the cells.

2. The method of claim 1, wherein the applied light is red light or near-infrared window light.

3. The method of claim 1, wherein applying the light to control biological behavior in the modified cell causes the modified cells to proliferate.

4. The method of claim 1, wherein applying the light control biological behavior in the modified cell causes activation of desired gene expression in the modified cells.

5. The method of claim 1, wherein an immune system response is suppressed at the localized area of the patient where the modified cells are exposed to the light of a certain wavelength.

6. The method of claim 1, wherein the optogenetic system comprises a protein and a biliverdin IXα chromophore.

7. The method of claim 6, wherein the protein comprises a photosensory domain and an enzymatic domain.

8. The method of claim 1, wherein applying the light to control biological behavior in the modified cell produces cyclic adenosine monophosphate in the optogenetic system.

9. The method of claim 1, wherein applying the light to express the one or more genetic elements in the modified cells produces cyclic-di-guanosine monophosphate in the optogenetic system.

10. The method of claim 9, wherein the optogenetic system comprises one or more cyclic-di-guanosine monophosphate sensitive transcription factors.

11. The method of claim 1, wherein the light of the certain wavelength is applied a plurality of times to the localized area over a period of time.

12. The method of claim 1, wherein the extracted cells are selected from a group consisting of regulatory T-cells, mesenchymal stromal cells, antigen presenting cells, Schwann cells, M2-type macrophages, and stem-like precursors of immunosuppressive cells.

13. A method of optogenetically modifying cells, comprising:
binding a chromophore to a protein in immunosuppressive cells, the protein comprising a photosensory domain and an enzymatic domain;
applying light of a certain wavelength to the cells to increase enzyme activity in the cells, the increased enzyme activity producing cyclic adenosine monophosphate in the cells;
placing cyclic adenosine monophosphate responsive elements in a promoter sequence of the cells; and
binding cyclic adenosine monophosphate sensitive transcription factors to the cyclic adenosine monophosphate responsive elements to alter expression of one or more genetic elements.

14. The method of claim 13, wherein binding cyclic adenosine monophosphate sensitive transcription factors to the cyclic adenosine monophosphate responsive elements alters the expression of one or more genetic elements.

15. The method of claim 13, wherein the cells are selected from a group consisting of regulatory T-cells, mesenchymal stromal cells, antigen presenting cells, Schwann cells, M2-type macrophages, and stem-like precursors of immunosuppressive cells.

16. The method of claim 13, wherein the cells are implanted into the patient for localized immunosuppression following the binding of the cyclic adenosine monophosphate sensitive transcription factors to the cyclic adenosine monophosphate responsive elements.

17. A method of optogenetically modifying cells, comprising:
extracting immunosuppressive cells or stem-like precursors of immunosuppressive cells from a patient;
binding a chromophore to a protein in the cells, the protein comprising a photosensory domain and an enzymatic domain;
applying light of a certain wavelength to the cells to increase enzyme activity in the, the increased enzyme activity producing cyclic-di-guanosine monophosphate in the cells;
placing cyclic-di-guanosine monophosphate responsive elements in a promoter sequence of the cells; and
binding cyclic-di-guanosine monophosphate sensitive transcription factors to the cyclic-di-guanosine monophosphate responsive elements to alter expression of one or more genetic elements.

18. The method of claim 17, wherein binding cyclic-di-guanosine monophosphate sensitive transcription factors to the cyclic-di-guanosine monophosphate responsive elements alters the expression of one or more genetic elements.

19. The method of claim 17, wherein the cells are selected from a group consisting of regulatory T-cells, mesenchymal stromal cells, antigen presenting cells, Schwann cells, M2-type macrophages and stem-like precursors of immunosuppressive cells.

20. The method of claim 17, wherein the cells are implanted into a localized area of the patient for localized immunosuppression following the binding of the cyclic-di-guanosine monophosphate sensitive transcription factors to the cyclic-di-guanosine monophosphate responsive elements.
